SEQUENCE LISTING (9 sequences)

SEQ ID NO: 1 - protein, 118 aa, Artificial Sequence
  mat3-VH
EVQLLESGGGLVQPGGSLRLSCAASGFTFSSYWMHWVRQAPGKGLEWVSDIARLSSYTNYADSVKGRFTISRDNSKNTLYLQMNSLRAEDTAVYYCARGLDARRMDYWGQGTLVTVSS

SEQ ID NO: 2 - protein, 113 aa, Artificial Sequence
  mat3-VL
QSVLTQPPSASGTPGQRVTISCTGSSSNIGAGYVVHWYQQLPGTAPKLLIYRNNQRPSGVPDRFSGSKSGTSASLAISGLRSEDEADYYCAAWDDSLNGWLFGGGTKLTVLGQ

SEQ ID NO: 3 - protein, 8 aa, Artificial Sequence
  HCDR1
FSSYWMHW

SEQ ID NO: 4 - protein, 19 aa, Artificial Sequence
  HCDR2
SDIARLSSYTNYADSVKGR

SEQ ID NO: 5 - protein, 11 aa, Artificial Sequence
  HCDR3
ARGLDARRMDY

SEQ ID NO: 6 - protein, 14 aa, Artificial Sequence
  LCDR1
TGSSSNIGAGYVVH

SEQ ID NO: 7 - protein, 7 aa, Artificial Sequence
  LCDR2
RNNQRPS

SEQ ID NO: 8 - protein, 12 aa, Artificial Sequence
  LCDR3
CAAWDDSLNGWL

SEQ ID NO: 9 - protein, 16 aa, Artificial Sequence
  LCDR1
SCTGSSSNIGAGYVVH

(19) United States
(12) Patent Application Publication
(10) Pub. No.: US 2021/0128728 A1
(43) Pub. Date: May 6, 2021

(54) PROLACTIN RECEPTOR ANTIBODY FOR MALE AND FEMALE PATTERN HAIR LOSS

(71) Applicant: Bayer Pharma Aktiengesellschaft, Berlin (DE)

(72) Inventors: Ekkehard MAY, Berlin (DE); Stefan Joachim JODL, Kleinmachnow (DE); Jörn KRÄTZSCHMAR, Berlin (DE); Christiane OTTO, Wuppertal (DE)

(73) Assignee: Bayer Pharma Aktiengesellschaft, Berlin (DE)

(21) Appl. No.: 16/629,499

(22) PCT Filed: Jul. 3, 2018
(86) PCT No.: PCT/EP2018/067912

§ 371 Date: Jan. 8, 2020

(30) Foreign Application Priority Data
Jul. 10, 2017 (EP) 17180554.2

(51) Int. Cl. A61K 39/395; C07K 16/28; A61K 47/18; A61K 47/22; A61K 47/20; A61K 47/26

(52) U.S. Cl. CPC A61K 39/39591; C07K 16/2869; A61K 47/183; A61K 2039/505; A61K 47/20; A61K 47/26; C07K 2317/76; A61K 47/22

(57) ABSTRACT

The present invention is related to a formulation of the prolactin receptor antibody mat3 and its use in the treatment of male and female pattern hair loss.

The present invention is directed towards a formulation of the prolactin receptor (PRLR) antibody mat3, as well as its use for the treatment of male and female pattern hair loss and prolactin-related forms of hair loss e.g. caused by prolactinoma, prolactin-increasing drugs such as neuroleptics or peripartal hair loss which is associated with high systemic prolactin levels.

### BACKGROUND OF THE INVENTION

Male and female pattern hair loss (M/FPHL) partly also described as “Androgenetic Alopecia” is the most common cause of hair loss and concerns up to 70% of males and 40% of females (Wolff, H. et al., (2016), Diagnostik und Therapie von Haar und Kopfhauterkrankungen. Deutsches Arzteblatt, 113, 377-386). M/FPHL affects primarily the top and front of the scalp whereby males have a receding hairline and females show thinning of the hair. It is believed that the male hormone dihydrotestosterone is a major player in male pattern hair loss. The pattern of hair loss in women differs from male-pattern baldness. In women, the hairline does not recede and does not lead to total baldness.

Scalp hair growth in cycles: the anagen phase is characterized by active hair growth; the catagen phase shows involution and is followed by the telogen phase (resting). The exogen phase (the release of the dead hair) coincides with the end of the telogen phase. Hair loss can be the consequence of disturbed hair growth in any phase.

Telogen hair loss can have many triggers (physiological and emotional stress, medical conditions, iron and zinc deficiency). Importantly M/FPHL in its early stages shows telogen hair shedding (Cleveland Clinic Journal of Medicine (2009). 76, 361-367). Anagen hair loss is often the consequence of radiation or chemotherapy.

Although some medicaments for treating M/FPHL are available such as, finasteride and minoxidil, treatment of hair loss is still an unmet medical need. Glucocorticoids are used for the treatment of alopecia areata, in which the body attacks its own anagen hair follicles and suppresses or stops hair growth. Minoxidil and finasteride are used for the treatment of M/FPHL loss in men whereas only minoxidil is also used for this indication in women. In general, all of these treatments have side-effects. Finasteride may lead to libido loss and impotence in men and other severe side effects like breast cancer are discussed; it is contraindicated in women. Minoxidil may lead to hypostatic reactions, angina pectoris and unwanted hair growth. Also not all patients treated are effectively treated with any of these drugs. Therefore the problem of treating M/FPHL has clearly not been solved and a significant unmet medical need remains yet.

In rodents, shaving experiments in adult animals were used to analyze the effects of compounds on hair loss by analyzing hair regrowth in the shaved area as readout paradigm (British Journal of dermatology (2008).159, 300-305). Shaving of adult animals at a time point in which hair growth is mostly resting (telogen phase), induces the anagen phase that is characterized by hair growth. It could be demonstrated that dihydrotestosterone application in wildtype mice delays hair regrowth after shaving whereas androgen receptor-deficient mice show enhanced regrowth after shaving (British Journal of dermatology 2008; 159:300-305).

Notably, all drugs that show efficacy in human androgenetic hair loss are effective in these shaving experiments, i.e. finasteride, minoxidil have been shown to stimulate hair regrowth in shaved wildtype mice (Int J Pharmaceutics (2005). 306, 91-98;Med Chem Lett (2007). 17, 5983-5988).

Prolactin (PRL) is a polypeptide hormone composed of 199 amino acids. PRL belongs to the growth hormone (GH), placental lactogen (PL) family of polypeptide hormones and is synthesized in lactotroph cells of the pituitary and in several extrapituitary tissues such as lymphocytes, mammary epithelial cells, the myometrium, and the prostate. Two different promoters regulate pituitary and extrapituitary PRL synthesis (BioEssays 28:1051-1055, 2006).

PRL binds to the PRL receptor (PRLR), a single transmembrane receptor belonging to the class 1 cytokine receptor superfamily (Endocrine Reviews 19:225-268, 1998). Upon ligand binding to pre-dimerized PRLRs, JAKs (predominantly JAK2, Janus Kinase 2) associated with the receptor, transphosphorylate and activate each other. In addition the PRLR is also phosphorylated and can bind to SH2-domain containing proteins such as STATs (Signal transducers and activators of transcription). Receptor bound STATs are subsequently phosphorylated, dissociate from the receptor and translocate to the nucleus where they stimulate transcription of target genes. In addition, activation of the Ras-Raf-MAPK pathway and activation of the cytoplasmic src kinase by PRLRs have been described (Endocrine Reviews 19: 225-268, 1998).

PRLR-mediated signaling plays a role in a variety of processes such as mammary gland development, lactation, reproduction, mammary and prostate tumor growth, autoimmune diseases, general growth and metabolism, and immunomodulation (Endocrine Reviews 19: 225-268, 1998; Annu. Rev. Physiol. 64: 47-67, 2002).

The pituitary PRL secretion can be inhibited by use of bromocriptine and other dopamine receptor 2 agonists (Nature Clinical Practice Endocrinology and Metabolism 2(10): 571-581, 2006). These agents, however, do not suppress extrapituitary PRL synthesis that can compensate successfully for the inhibition of pituitary PRL synthesis leading to almost unimpaired PRLR-mediated signaling (Endocrine Reviews 19:225-268, 1998). Therefore it is not surprising that dopamine type 2 receptor agonists were not beneficial in patients suffering from breast cancer or autoimmune diseases such as systemic lupus or rheumatoid arthritis (Breast Cancer Res. Treat. 14:289-29, 1989; Lupus 7:414-419, 1998) although prolactin has been implicated in these diseases. Local prolactin synthesis in breast cancer cells or lymphocytes which plays a pivotal role in mammary carcinoma or autoimmune diseases, respectively, was not blocked by dopamine receptor agonists. PRLR-deficient mice similar to androgen-receptor-deficient mice show enhanced hair growth whereas prolactin administration to wildtype mice delays hair growth (J Endocrinol (2006).191, 415-425).

Foitzik reported (Am J Pathol (2003), 162 (5), 1611-1621) that prolactin may have an effect on hair growth. Based on in vitro analysis of hair growth after incubation with unphysiologically high prolactin concentrations Foitzik concluded that PRLR antagonists could be useful for the treatment of androgenic alopecia. However, human PRL can interact with the human PRL receptor as well as the growth hormone receptor (GHR). By using PRL at high doses it is unclear, which receptor is activated, i.e. the GHR or the PRLR. It was neither specified nor disclosed which kind of PRLR antagonists were employed in the study. PRL variants as well as competitive PRLR antagonists are not effective in neutralizing local PRL signaling in the hair follicle due to their negative characteristics which are 1) a reduced PRLR inhibition in the presence of increasing PRL concentrations due to the competitive mechanism of action, 2) reduced half-life, and 3) reduced affinity to the PRLR if compared to PRL.

Recently several PRLR-antibodies which interfere with PRLR-mediated signaling have been described (WO2012163932, WO2011069799, WO2011069795). It has been demonstrated that neutralising PRLR antibodies (WO2011069799) stimulate hair regrowth in shaved normo- and hyperprolactinemic mice. It could also be demonstrated that the prolactin receptor (PRLR) is expressed in hair follicles in the skin of monkeys and mice (see example 1) and that the PRLR is neutralized by binding of the PRLR-specific antibody 005-004. It could also be shown that the PRLR antibody 005-004 stimulates hair regrowth in shaved female mice (Otto, C. et al. (2015) Endocrinology 156 (11), 4365-4373). The described specific PRLR antibodies do not interact with the GHR. Blockade of the PRLR enhances hair regrowth in mice as it has been demonstrated with anti-androgens in the same animal model. However the fur on the mice back cannot be considered as a relevant model for human scalp hair. Therefore the described effect on mouse fur hair is merely suggestive but not conclusive per se regarding potential treatment effects in human alopecia. However, even though mouse data are conclusive and consistent with monkey data in the androgen receptor and PRLR context, translation of mouse data to the human situation may be hampered by the fact that re-growth of mouse trunk hair (fur) may differ from scalp hair re-growth in monkey and human as well as by the fact that hair re-growth after spontaneous hair loss in humans and monkeys may differ from re-growth after shaving of mouse trunks.

There is still an unmet medical need for the provision of medicaments for the treatment of M/FPHL. The underlying problem of the present invention lies in the provision of a new medicament which can be used in the treatment of M/FPHL.

### SUMMARY OF THE INVENTION

This invention is related to a stable antibody-containing formulation of a PRLR antibody which is surprisingly effective in the most accepted non-human primate model for M/FPHL (the stumptail macaque model) and is thus promising as a new treatment also for human M/FPHL.

Recent studies employing this monkey model of spontaneous post-puberty scalp hair loss with high predictive value for human male and female pattern hair loss demonstrated the efficacy of the PRLR antibody mat3 when animals were treated with a stable antibody-containing formulation. The antibody itself is subject matter of the PCT application WO2012163932 whereas the formulation is disclosed in the PCT application WO2014036076, each of which is incorporated by reference herein in their entirety.

Employing stumptail macaques it could be demonstrated that the treatment with the stable PRLR antibody-containing formulation will likely be efficacious in human male and female pattern hair loss. A very high proportion (81%) of animals (9 out of 11) responded to compound treatment, although the population was heterogeneous with respect to age and gender and the observed area showed long-term baldness before treatment. 5 out of 11 monkeys were >25 years of age and were considered particularly difficult to treat. However, three of those responded to compound treatment. Notably, completely bald scalp areas responded best. The PRLR antibody mat3 showed more efficacy and was effective following a more convenient regime (s.c. twice per month) than the only two approved medications for M/FPHL (minoxidil (topical, daily), and finasteride (oral, once daily, men only). (See example 4 and FIGS. 2-3)).

Therefore the stable PRLR antibody mat3-containing formulation can be considered as a new treatment option for M/FPHL in women and men. The provision of a specific stable antibody mat3-containing formulation solves the underlying problem of providing a new medicament for M/FPHL in women and men. The formulation can also be employed for the treatment of prolactin related forms of hair loss e.g. caused by prolactinoma, prolactin-increasing drugs such as neuroleptics or peripartal hair loss which is associated with high systemic prolactin levels.

### DESCRIPTION OF THE FIGURES

FIG. 1: Immunoreactivity for the PRLR in mammary glands and skin from female mice and cynomolgus monkeys

FIG. 1A: skin female cynomolgus monkey; FIG. 1B: mammary gland female cynomolgus monkey; FIG. 1C: skin, female mouse, FIG. 1D: mammary gland female mouse.

Immunoreactivity for the PRLR can be found in hair follicles and epidermal epithelial cells in the skin of female mice (FIG. 1C) and monkeys (FIG. 1A). Strong immunoreactivity for the PRLR was demonstrated in mammary epithelial cells from both species (FIG. 1B and 1D). These experiments provide evidence for a role of PRLR-mediated signaling in hair follicle biology.

FIG. 2: Comparison of hair growth: Historical data with Minoxidil and Finasteride vs Treatment with PRLR antibody mat 3

FIG. 2 represents an analysis of the effects of the approved reference compounds minoxidil and finasteride on hair growth (FIG. 2A) in comparison to the treatment with the novel PRLR antibody mat3 (FIG. 2B), both studies employed the stumptail macaque model.

FIG. 2A: In the study employing minoxidil and finasteride the change in hair weight in mg/in² was measured over a period of 20 weeks (open squares: finasteride in combination with vehicle, filled squares: finasteride in combination with minoxidil; circles minoxidil alone). During the time of treatment a plateau was reached after approximately 12 weeks followed by decay in hair weight. (from: Hair Growth Effects of Oral Adinistration of Finasteride, a Steroid 5a-Reductase Inhibitor,

Alone and in a Combination with Topical Minoxidil in the Balding Stumptail Macaque. Diani, A.R. et al. (1992). Journal of Clinical Endocrinology and Metabolism, 74, 345-350).

FIG. 2B: In the study in which stumptail macaques were treated with the PRLR antibody mat3 the percentage of thick hair was measured over a treatment period of 6 months. FIG. 2B shows that no plateau was reached during this time. These data show the superior effect of the PRLR antibody mat3 on terminal hair regrowth in comparison with the reference compounds minoxidil and finasteride. An increase of the fraction of terminal hairs in the bald area could be observed in female (squares) and in male (diamonds) animals. The range of increase was 50-220 hairs/cm², younger animals responded better than senile animals. (filled squares: females, filled diamonds: male animals).

FIG. 3: Terminal (“thick”) hair count

FIG. 3 represents an analysis of terminal hair counts in the “bald” (A), “transition” (B), “rear head” (C) and “trunk” (D) areas over the treatment period of 24 weeks. Such areas were predefined at baseline and marked by 2 tattoos which occurred in the upper left and lower right corner of the trichoscan image and allowed to monitor the very same hair follicles over the entire study duration. A drastic increase of the terminal hair count in bald areas (109% increase) was demonstrated as well as an increase in transition areas (27%). Expectedly, minor changes occurred in the rear head and trunk areas where few vellus hairs but many terminal hairs were present already before treatment start.

### DETAILED DESCRIPTION OF THE INVENTION

The present invention is based on the discovery that the stable prolactin receptor antibody mat3-containing formulation promotes hair growth and can therefore deliver a therapeutic benefit to a subject.

### Definitions

As described above, the present disclosure provides a PRLR antibody formulation that stabilizes the antibody in liquid form or in lyophilized form at intended storage conditions. The formulation described herein includes one or more pharmaceutically acceptable excipients or stabilizers, and is contained in buffered media at a suitable pH and is substantially isosmotic with physiological fluids. For systemic administration, injection is one possible route of administration, including intramuscular, intravenous, intraperitoneal, and subcutaneous for injection. Because of their low viscosity, the presently disclosed PRLR antibody formulation can be conveniently processed via, for example, ultrafiltration and sterile filtration and can be administered to a patient via injection, including both intravenous and subcutaneous injection. Moreover, because they are substantially isosmotic, the presently disclosed PRLR antibody formulation reduces tissue damage or other adverse physiologic effects and thereby achieving favorable patient tolerance and increased patient compliance

The formulation described herein is characterized by the substantial absence of added salt other than an organic salt or an inorganic salt that is used to buffer the formulation, which provides the flexibility for increasing the concentrations of other stabilizers, such as sucrose, while maintaining the osmolality of the formulation for improved in vivo tolerability and, consequently, increased patient compliance. Moreover, the low viscosity of the presently described formulation permits convenient processing, including ultrafiltration and sterile filtration, and injection of the drug product solution through the needle.

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.” Furthermore, where the description of one or more embodiments uses the term “comprising,” those skilled in the art would understand that, in some specific instances, the embodiment or embodiments can be alternatively described using the language “consisting essentially of” and/or “consisting of”

As used herein, the term “viscosity” refers to the resistance of a liquid formulation to flow, such as when injected through a syringe needle during administration to a patient. Viscosity measurements can be done by a cone and plate technique with a Peltier element set at a defined temperature, such as 22° C.-23° C. as described herein. Typically, a well-defined shear stress gradient is applied to the liquid formulation and the resulting shear rate is measured. The viscosity is the ratio of the shear stress to the shear rate. As used herein, viscosity is expressed in units of mPa-S at 22° C.-23° C. wherein 1 mPa-S=1 cP. The high concentration, low viscosity, substantially isosmotic formulations disclosed herein are typically characterized by having a viscosity ranging from 1 to 8 mPa-S at 22° C-23° C.

As used herein, the term “osmolality” refers to a measure of solute concentration, defined as the number of mmole of solute per kg of solution. A desired level of osmolality can be achieved by the addition of one or more stabilizer such as a sugar or a sugar alcohol including mannitol, dextrose, glucose, trehalose, and/or sucrose. Additional stabilizers that are suitable for providing osmolality are described in references such as the handbook of Pharmaceutical Excipients (Fourth Edition, Royal Pharmaceutical Society of Great Britain, Science & Practice Publishers) or Remingtons: The Science and Practice of Pharmacy (Nineteenth Edition, Mack Publishing Company).

As used herein, the term “about” refers to +/−10% of the unit value provided. As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term substantially is therefore used herein to capture the potential lack of completeness inherent in many biological and chemical phenomena.

As used herein, the terms “isosmotic” and “isotonic” are used interchangeably with the terms “substantially isosmotic,” and “substantially isotonic” and refer to formulations characterized by having an osmotic pressure that is the same as or at least substantially equivalent to the osmotic pressure of another solution, which is achieved by formulations wherein the total concentration of solutes, including both permeable and impermeable solutes, in the formulation are the same as or at least substantially equivalent to the total number of solutes in another solution. Thus, while it will be appreciated by those of skill in the art that “isosmotic” and “isotonic” formulations that are used for in vivo administration generally have an osmolality ranging from about 270 mmol/kg to about 310 mmol/kg, in the context of the high concentration, low viscosity formulations of the present disclosure, the terms “isosmotic,” “isotonic,” “substantially isosmotic,” and “substantially isotonic” are used interchangeably to refer to formulations having an osmolality ranging from about 240 mmol/kg to about 380 mmol/kg, or from about 270 mmol/kg to about 370 mmol/kg, or from about 300 mmol/kg to about 330 mmol/kg.

The presently disclosed high concentration, low viscosity, substantially isosmotic PRLR antibody formulations contain from about 0 mM to about 70 mM histidine; from about 50 ppm to about 300 ppm of a non-ionic surfactant such as, for example, polysorbate (Tween®) 80 and/or polysorbate (Tween®) 20; from about 34 mM to about 292 mM of a sugar or sugar alcohol, such as, for example, mannitol, dextrose, glucose, trehalose, and/or sucrose; from about 0 mM to about 50 mM arginine; from about 0 mM to about 50 mM Iysine; from about 0 mM to about 270 mM glycine or alanine; from about 0 mM to about 10 mM methionine; and from about 1 mg/ml to about 150 mg/ml of the PRLR antibody at a pH from about pH 5.0 to about pH 6.5. The formulations disclosed herein exhibit a viscosity ranging from about 1 to about 8 mPa-S at 22° C-23° C. and osmolality ranging from about 240 to about 380 mmol/kg.

In these formulations, histidine is a buffer agent, which can be used to maintain the formulation pH from about pH 5.0 to about pH 6.5, or from about pH 5.5 to about pH 6.0, such as about pH 5.0, about pH 5.5, about pH 6.0, or about pH 6.5.

Sugars or sugar alcohol, such as mannitol, dextrose, glucose, trehalose, and/or sucrose, are used separately or in combination both as cryo-protectants and a stabilizer the PRLR antibody in liquid formulations as well as during lyophilization.

Non-ionic surfactants such as polysorbates, including polysorbate 20 and polysorbate 80;

polyoxamers, including poloxamer 184 and 188; pluronic® polyols; and other ethylene/polypropylene block polymers, stabilize the PRLR antibody during processing and storage by reducing interfacial interaction and prevent antibody from adsorption.

Arginine is a protein solubilizer and also a stabilizer that reduces antibody and other protein aggregation, such as the PRLR antibody aggregation, and other possible degradation. Methionine is an antioxidant that prevents antibody oxidation during processing and storage.

Sugars and inorganic salts are commonly used as protein stabilizers; however, both sugars and inorganic salts are also effective tonicity agents. If a formulation requires a high concentration of one or more sugars to stabilize the PRLR antibody, the inorganic salt concentration should be zero or kept very low in order to maintain the formulation's osmolality such that injection pain is reduced upon administration.

As used herein, the term “salt” refers to inorganic salts, which include sodium chloride (NaCl), sodium sulfate (Na₂S0₄), sodium thiocyanate (NaSCN), magnesium chloride (MgCl), magnesium sulfate (MgS0₄), ammonium thiocyanate (NH₄SCN), ammonium sulfate ((NH₄)2504), ammonium chloride (NH4C1), calcium chloride (CaCl₂), calcium sulfate (CaS0₄), zinc chloride (ZnCl₂) and the like, or combinations thereof The PRLR antibody formulation disclosed herein is characterized by a substantial absence of added salt and are, therefore, referred to herein as a salt-free antibody formulation. It will be understood by those of skill in the art that the presence of inorganic salts within the presently disclosed formulations that are introduced by pH adjustment are not considered to be added salts. Such inorganic salts when introduced by pH adjustments, if present in a formulation according to the present disclosure, should not exceed a concentration of about 2 mM.

As used herein, the term “surfactant” includes non-ionic surfactants including, without limitation, polysorbates, such as polysorbate 20 or 80, and the polyoxamers, such as polyoxamer 184 or 188, pluronic® polyols, and other ethylene/polypropylene block polymers. Amounts of surfactants effective to provide stable high concentration the PRLR antibody formulation are usually in the range of 50 ppm to 300 ppm. The use of non-ionic surfactants permits the formulation to be exposed to shear and surface stresses without causing denaturation of the PRLR antibody, and also reduce the adsorption on the surfaces during processing and storage. The formulation disclosed herein include, without limitation, a formulation having one or more non-ionic surfactant(s) including, for example, one or more polysorbate(s), such as polysorbate 20 or 80; one or more polyoxamers, such as poloxamer 184 or 188; pluronic® polyols; and/or one or more ethylene/polypropylene block polymer(s). Exemplified herein are formulations having a polysorbate, such as polysorbate 20 (Tween® 20) or 10 polysorbate 80 (Tween® 80).

The term “vellus hair” refers to short, thin, slight-colored and barely noticeable thin hair that develops during childhood. Each strand of vellus hair is usually less than 2 mm long.

The term “terminal hair” refers to thick, long and pigmented hair, usually in the anagen phase of hair growth. Terminal hair is defined to have an average diameter of 31.5 μm or more.

The term “bald area” refers to a predefined region at the central/frontal part of the scalp that was initially hairy yet affected by complete loss of visible, e.g. terminal hairs. Follicles remain producing thin (generally vellus-type) hairs.

The term “transition area” refers to a predefined region of the scalp where visually obvious, however, incomplete hair loss has occurred, i.e. terminal hair density is reduced as compared to the rear scalp area while fallen-off terminal hairs are now replaced by vellus-type hairs.

The term “rear head area” refers to the part of the scalp where no hair loss has occurred, i.e. the vast majority of hairs belong to the terminal hair type and only a few vellus-type hairs are present.

The term “trunk” refers to a predefined area at the flank of the monkey's back which is covered by fur hairs, i.e. primarily thick terminal hairs.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “an antibody that binds to PRLR” refers to an antibody that is capable of binding PRLR with sufficient affinity such that the antibody is useful as a diagnostic and/or therapeutic agent in targeting

PRLR.

As used herein, the term “antibody” refers to a class of proteins that are generally known as immunoglobulins. Antibodies include full-length monoclonal antibodies (mAb), such as IgG2 monoclonal antibodies, which include immunoglobulin Fc regions. The term antibody also includes bispecific antibodies, diabodies, single-chain molecules, and antibody fragments such as Fab, F(ab′)₂, and Fv. The antibody preferably comprised of four polypeptide chains, two heavy (H) chains and two light (L) chains which are typically inter-connected by disulfide bonds. Each heavy chain is comprised of a heavy chain variable region (abbreviated herein as VH) and a heavy chain constant region. The heavy chain constant region can comprise e.g. three domains CH1, CH2 and CH3. Each light chain is comprised of a light chain variable region (abbreviated herein as VL) and a light chain constant region. The light chain constant region is comprised of one domain (CL). The VH and VL regions can be further subdivided into regions of hypervariability, termed complementarity determining regions (CDR), interspersed with regions that are more conserved, termed framework regions (FR). Each VH and VL is typically composed of three CDRs. As used herein, the term “anti-PRLR antibody” refers to an antibody having binding specificity against the human PRLR protein as well as fragments and variants of the human PRLR protein. Anti-PRLR antibodies presented herein can be IgG2 antibodies and include anti-PRLR IgG2 monoclonal antibodies, such as chimeric, humanized, and fully-human anti-PRLR IgG2 monoclonal antibodies. Anti-PRLR monoclonal antibodies, including full-length antibodies and antigen binding fragments and variants thereof, that are suitable for use in the formulations disclosed herein are presented in PCT Patent Publication numbers. WO/20111069799,

WO/20111069795, and WO/2012163932, each of which are incorporated by reference herein in their entirety. This includes also the PRLR antibody mat3 which comprises an antigen binding domain, wherein said antigen binding domain comprises a heavy chain variable region and a light chain binding variable region, wherein
a. the amino acid sequences of the HCDR1, HCDR2, and HCDR3 of said heavy chain variable region are selected from the group consisting of the amino acid sequence of SEQ ID NO: 3, 4, and 5 respectively, and
b. the amino acid sequences of the LCDR1, LCDR2, and LCDR3 of said light chain variable region are selected from the group consisting SEQ ID NO: 6, 7, and 8.

The amino acid sequences of the LCDR1 can also be depicted by SEQ ID NO: 9. The PRLR antibody comprises a heavy chain variable region and a light chain binding variable region, wherein the amino acid sequence of said heavy chain variable region is according to SEQ ID NO: 1, and the amino acid sequence of said light chain variable region is according to SEQ ID NO: 2.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the term “monoclonal” indicates the character of the antibody as not being a mixture of discrete antibodies. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins. The term “monoclonal” is not to be construed as to require production of the antibody by any particular method. For example, the monoclonal antibodies to be used may be made by the hybridoma method first described by Kohler et al., Nature, 256: 495 [1975, or may be made by recombinant DNA methods (see, e.g., U.S. Pat. No. 4,816,567). Monoclonal antibodies can also be isolated from phage display libraries using the techniques such as those described in Clackson et al., Nature 352:624-628 (1991) and Markset al., J Mol. Biol. 222:581-597 (1991). The “monoclonal antibodies” may also be recombinant, chimeric, humanized, human, Human Engineered™, or antibody fragments, for example.

Monoclonal antibodies include “chimeric monoclonal antibodies” wherein a portion of a heavy and/or light chain includes sequences from antibodies derived from one species, while the remainder of the antibody, including the Fc region, includes sequences from antibodies derived from a second species, and the second species may be human. See, e.g., U.S. Patent No. 4,816,567 and Morrison et al., Proc. Natl. Acad. Sci. USA 81:6851-6855 (1984).

Monoclonal antibodies also include “humanized monoclonal antibodies” wherein one or more complementarity determining region (CDR) from a heavy and/or light chain sequence from antibodies derived from one species replace one or more CDR from a heavy and/or light chain sequence from antibodies derived from a second species, and the second species may be human. The process of “humanization” is usually applied to monoclonal antibodies developed for administration to humans. See, e.g., Riechmann et al., Nature 332(6162):323-27 (1988) and Queen et al., Proc. Natl. Acad. Sci. USA 86(24):10029-33 (1989).

Monoclonal antibodies also include “fully-human monoclonal antibodies” wherein the entire heavy and light chain sequences are derived from human antibody sequences. Fully-human monoclonal antibodies can be generated by phage display technologies and can be isolated from mice that have been genetically engineered to express the human antibody repertoire. See, e.g., McCafferty et al., Nature 348(6301):552-554 (1990), Marks et al., J Mol. Biol. 222(3):581-597 (1991), and Carmen and

Jermutus, Brief Funct. Genomic Proteomic 1(2): 189-203 (2002).

As used herein, an antibody “binds specifically to”, is “specific to/for” or “specifically recognizes” an antigen of interest, e.g. PRLR polypeptide antigen target, is one that binds the antigen with sufficient affinity such that the antibody is useful as a therapeutic agent in targeting a cell or tissue expressing the antigen, and does not significantly cross-react with other proteins or does not significantly cross-react with proteins other than orthologs and variants (e.g. mutant forms, splice variants, or proteolytically truncated forms) of the aforementioned antigen target. The term “specifically recognizes” or “binds specifically to” or is “specific to/for” a particular polypeptide or an epitope on a particular polypeptide target as used herein can be exhibited, for example, by an antibody, or antigen-binding fragment thereof, having a monovalent K_{D} for the antigen of less than about 10⁻⁴ M, alternatively less than about 10⁻⁵ M, alternatively less than about 10⁻⁶ M, alternatively less than about 10⁻⁷ M, alternatively less than about 10⁻⁸M, alternatively less than about 10⁻⁹ M, alternatively less than about 10⁻¹⁰ M, alternatively less than about 10⁻¹¹ M, alternatively less than about 10⁻¹² M, or less. An antibody “binds specifically to,” is “specific to/for” or “specifically recognizes” an antigen if such antibody is able to discriminate between such antigen and one or more reference antigen(s). In its most general form, “specific binding”, “binds specifically to”, is “specific to/for” or “specifically recognizes” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out.

“Binding affinity” or “affinity” refers to the strength of the total sum of non-covalent interactions between a single binding site of a molecule and its binding partner. Unless indicated otherwise, as used herein, “binding affinity” refers to intrinsic binding affinity which reflects a 1:1 interaction between members of a binding pair (e.g. an antibody and an antigen). The dissociation constant “K_{D}” is commonly used to describe the affinity between a molecule (such as an antibody) and its binding partner (such as an antigen) i.e. how tightly a ligand binds to a particular protein. Ligand-protein affinities are influenced by non-covalent intermolecular interactions between the two molecules. Affinity can be measured by common methods known in the art, including those described herein. In one embodiment, the “K_{D}” or “K_{D} value” according to this invention is measured by using surface plasmon resonance assays using a Biacore T200 instrument (GE Healthcare Biacore, Inc.). Other suitable devices are BIACORE T100, BIACORE(R)-2000, BIACORe 4000, a BIACORE (R)-3000 (BlAcore, Inc., Piscataway, N.J.), or ProteOn XPR36 instrument (Bio-Rad Laboratories, Inc.).

As used herein the phrase “antibodies antagonize prolactin receptor mediated signaling” is meant to refer to a blockade of PRLR activation by the antibodies of the present invention which leads to an inhibition of PRLR mediated signaling.

An “antagonistic” antibody or a “blocking” antibody is one which significantly inhibits (either partially or completely) a biological activity of the antigen it binds.

The term “maturated antibodies” or “maturated antigen-binding fragments” such as maturated Fab variants includes derivatives of an antibody or antibody fragment exhibiting stronger binding—i. e. binding with increased affinity—to a given antigen such as the extracellular domain of a target protein. Maturation is the process of identifying a small number of mutations within the six CDRs of an antibody or antibody fragment leading to this affinity increase. The maturation process is the combination of molecular biology methods for introduction of mutations into the antibody and screening for identifying the improved binders.

As used herein, the term “pharmaceutically effective amount” of a PRLR antibody formulation refers to an amount of the formulation that provides therapeutic effect in an administration regimen, including alleviating some or all of such symptoms of disease or reducing the predisposition to the disease, when administered in accordance with the desired treatment regimen.

The high concentration PRLR antibody formulations disclosed herein typically include an anti-PRLR antibody at a concentration ranging from about 1 mg/ml to about 150 mg/ml, or from about 2 mg/ml to about 120 mg/ml, or from about 5 mg/ml to about 100 mg/ml, or from about 7.5 mg/ml to about 60 mg/ml. Within some aspects the concentration of anti PRLR antibody within these formulations is about 2 mg/ml, or about 7.5 mg/ml, or about 20 mg/ml, or about 50 mg/ml, or about 60 mg/ml. Such formulations are typically administered in a volume of less than about 2 ml, or about 1.5 ml, or about 1 ml, or about 0.5 ml per injection site for subcutaneous injection. Preferred is a formulation containing the PRLR antibody mat 3 which comprises an antigen binding domain, wherein said antigen binding domain comprises a heavy chain variable region and a light chain binding variable region, wherein
a. the amino acid sequences of the HCDR1, HCDR2, and HCDR3 of said heavy chain variable region are selected from the group consisting of the amino acid sequence of SEQ ID NO: 3, 4, and 5 respectively, and
b. the amino acid sequences of the LCDR1, LCDR2, and LCDR3 of said light chain variable region are selected from the group consisting SEQ ID NO: 6, 7, and 8.

The amino acid sequences of the LCDR1 can also be depicted by SEQ ID NO: 9. The PRLR antibody comprises a heavy chain variable region and a light chain binding variable region, wherein the amino acid sequence of said heavy chain variable region is according to SEQ ID NO: 1, and the amino acid sequence of said light chain variable region is according to SEQ ID NO: 2

The formulation can either be liquid or lyophilized and can be stable at ° C. for at least 6 months.

Within other aspects, the anti-PRLR antibody formulation contains about 5- 30 mM histidine, about 34-292 mM sucrose, about 50-150 ppm non-ionic surfactant, about 10-50 mM arginine, about 1-10 mM methionine, about 20-120 mg/ml anti-PRLR antibody at a pH ranging from about pH 5.0 to about pH 6.5, such as pH 5.5.

Within other aspects, the anti-PRLR antibody formulation contains about 5- 30 mM histidine, about 34-292 mM sucrose, about 50-150 ppm polysorbate, about 10-50 mM arginine, about 1-10 mM methionine, about 20-120 mg/ml anti-PRLR antibody at a pH ranging from about pH 5.0 to about pH 6.5, such as pH 5.5.

Within other aspects, the anti-PRLR antibody formulation contains about 10 mM histidine, about 234 mM sucrose, about 80 ppm polysorbate 80, about 30 mM arginine, about 5 mM methionine, about 60 (40) mg/ml anti-PRLR antibody at a pH ranging from about pH 5.0 to about pH 6.5, such as pH 5.5.

Within other aspects, the anti-PRLR antibody formulation contains about 1.8 mM L-histidine and 8.2 mM L-histidine HC1, about 234 mM sucrose, about 80 ppm polysorbate 80, about 30 mM L-arginine HC1, about 5 mM L-methionine, about 60 (40) mg/ml anti-PRLR antibody at a pH ranging from about pH 5.0 to about pH 6.5, such as pH 5.5.

The term “pharmaceutical formulation” / “pharmaceutical composition” refers to a preparation which is in such form as to permit the biological activity of an active ingredient contained therein to be effective, and which contains no additional components which are unacceptably toxic to a subject to which the formulation would be administered.

Thus, the present disclosure provides anti-PRLR mAb formulations, including anti-PRLR IgG2 mAb formulations, wherein the anit-PRLR mAb is soluble at high protein concentrations. The anti-PRLR mAb in the formulation disclosed herein remain soluble at concentrations of between about 1 mg/ml to about 150 mg/ml and remain stable under isosmotic storage conditions and exhibit reduced viscosity as compared to currently available antibody formulations.

The anti-PRLR antibody having the sequence as disclosed in the PCT applications WO2012163932 and WO2014036076, each of which is incorporated by reference in its entirety) is an IgG2 antibody that blocks prolactin receptor (PRLR). As mentioned above the PRLR antibody can induce hair growth by blocking PRLR, thereby overcoming deficiencies in M/FPHL. The high concentration, salt free anti-PRLR antibody formulations presented herein can be administrated to the patients via intravenous injection or subcutaneous injection or other injection routes.

### Antibody Generation

The antibody mat3 of the present invention and its generation are subject matter of the application WO2012163932. The disclosure of this application is incorporated herewith in its entirety. The sequences of the antibody mat3 are shown in table 1. The PRLR antibody formulation was prepared as disclosed in the application WO 2014036076. The disclosure of this application is incorporated herewith in its entirety.

TABLE 1
Sequences of the PRLR antibody mat3:

| Sequence region | Sequence Type | SEQ ID NO |
|---|---|---|
| Heavy chain | protein | 1 |
| variable region | | |
| Light chain | protein | 2 |
| variable region | | |
| HCDR1 | protein | 3 |
| HCDR2 | protein | 4 |
| HCDR3 | protein | 5 |
| LCDR1 | protein | 6 |
| LCDR2 | protein | 7 |
| LCDR3 | protein | 8 |
| LCDR1 | protein | 9 |

The use of the antibody mat3 in a given formulation for preventing M/FPHL is subject matter of the present invention. Also object of the present invention is the use of the formulation for the treatment of prolactin related forms of hair loss e.g. caused by prolactinoma, prolactin-increasing drugs such as neuroleptics or peripartal hair loss which is associated with high systemic prolactin levels.

### EXAMPLES

### Example 1

### Immunohistochemistry for the PRLR in Skin and Mammary Glands of Female Cynomolgus Monkeys and Mice

Immunohistochemistry was performed on paraffin-embedded sections of mammary glands and skin from a female cynomolgus monkey and female mice using a rabbit polyclonal anti PRLR antiserum from Santa Cruz directed against the c-terminus of the human PRLR (aa 323-622) (sc-20992). This antibody crossreacts with human, murine, and rat PRLR.

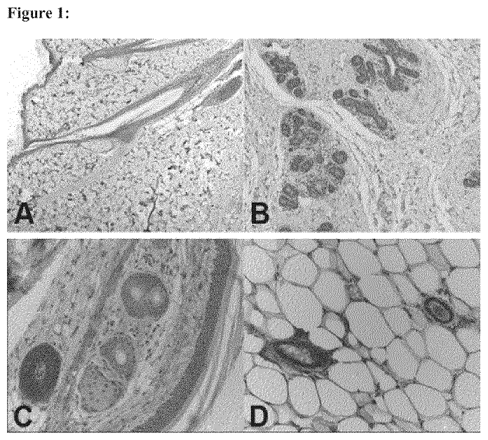

Immunoreactivity for the PRLR can be found in hair follicles and epidermal epithelial cells in the skin of female mice (FIG. 1C) and monkeys (FIG. 1A). Strong immunoreactivity was also observed as expected in mammary epithelial cells from both species (FIG. 1B and 1D). No immunoreactivity was observed, when the primary antibody was omitted.

These results provide evidence for a role of PRLR-mediated signaling in hair follicle biology.

### Example 2

### Neutralising PRLR Antibody mat3, Stimulates Hair Regrowth in Stumptail Macaques

The effects of the PRL antibody mat3 on hair regrowth in stumptail macaques were analysed. These animals are an accepted key model for human hair loss with a high predictive value. The studies were performed in the laboratory of the Institute of Molecular Medicine, Peking University, Yiheyuan Road, No. 5, 100871 Beijing, China. The group size was 11, whereby one senile female animal died in anaesthesia in week 16; the group consisted of 4 female animals and 7 male animals, age 12-27 years. The animals received 40 mg/kg PRLR antibody mat3 subcutaneously twice a month. The antibody solution is depicted in table 2:

TABLE 2
Composition of PRLR antibody mat3 s.c. solution

| Composition | Concentration | | Function |
|---|---|---|---|
| L-Arginine HCl | 30 | mM | Stabilizer |
| L-Histidine | 1.8 | mM | Buffer agent |
| L-Histidine HCl | 8.2 | mM | Buffer agent |
| L-Methionine | 5 | mM | Stabilizer (anti-oxidant) |
| Polysorbate 80 | 80 | ppm | Stabilizer (surfactant) |

| | | |
|---|---|---|
| Sucrose | 8% | Stabilizer |

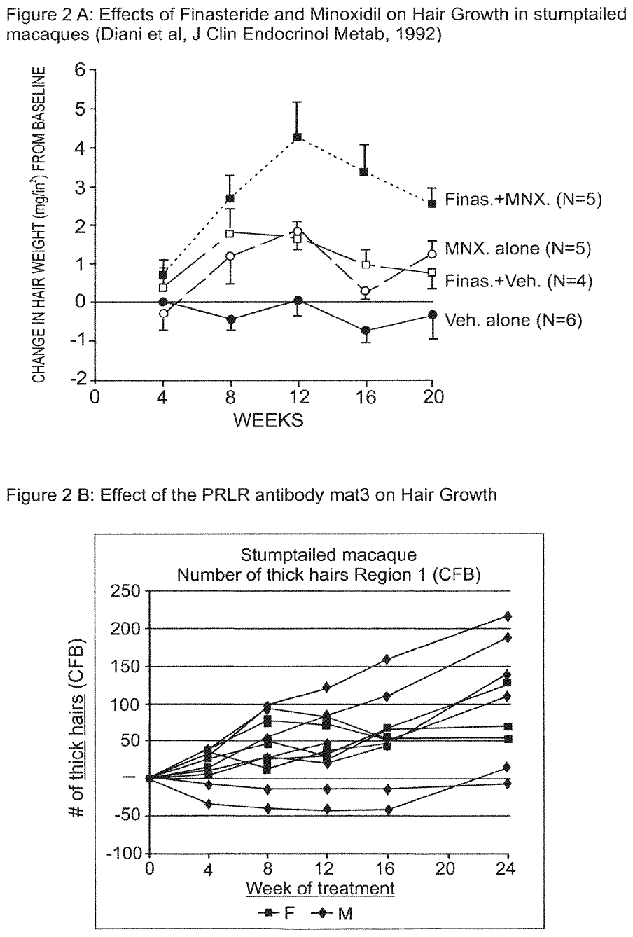

Hair regrowth was determined in the following areas: bald, transition, unaffected, trunk (fur). Biopsies from 2 adjacent transition areas were sampled as well as blood and serum. The primary readout was a phototrichogram, e.g. a trichoscan to determine terminal hair density. The baseline values were compared with the values obtained after every four weeks. The secondary readout was the standardized optical hair status, histology, and reactive prolactin levels. General parameters e.g. body weight, blood cell count were measured every month. It could be demonstrated that there was a robust and visible efficacy in bald and/or transition areas in comparison to the baseline data (see FIGS. 2). Even though the study was not designed as safety/tolerability/toxicology study, test animals were observed for behavioral and phenotypical anomalies. There were no apparent safety or tolerability signals during or after the treatment period related to drug administration.

Data for the trichoscans were collected at the following time points: baseline (starting day of treatment), 4-week treatment (d28), 8-week treatment (d56), 12-week treatment (d84), 16-week treatment (d112), 24-week treatment (d168) and at day 364, i.e. 24 weeks after the last dosing. The “best” phototrichoscan images (focus, contrast, quality of dyeing, absence of skinfolds) were chosen for each time point at each pre-defined area. At the individual time points, hair thicknesses were measured. Based on hair diameters, the number of vellus and terminal hairs per cm² was determined. Both measurements (hair diameters and count) were performed using Datinf TrichoScan Smart Software in semi-automated manner with manual curating. For consistency reasons each region was analyzed by the same observer over time. The obtained data showed an increased terminal hair count in the previously bald region in 9 out of 11 monkeys. The increase in hair count ranged from 50 - 220 hairs/cm² in responder monkeys (see FIG. 2B). The effect was observed in male and female monkeys. Younger animals responded better than seniles ones. No plateau of efficacy was reached in 6 months of treatment with the antibody mat3. Best effects with regard to increase of absolute numbers of terminal hairs as well as with regard to percentage increase was observed in previously bald areas, i.e. such areas where vellus hairs were dominating before treatment. Such areas had initially been considered the most difficult to treat since baldness of these areas was preexisting for decades in some of the monkeys. Six months after ceasing the treatment, no further increase of hair diameters or increase of terminal hairs has been observed. However, there was also only a marginal (insignificant) drop from the level reached at the end of the study. The proportion of terminal hairs was significantly higher than before start of treatment 12 months ago, i.e. a long lasting effect of treatment was observed.

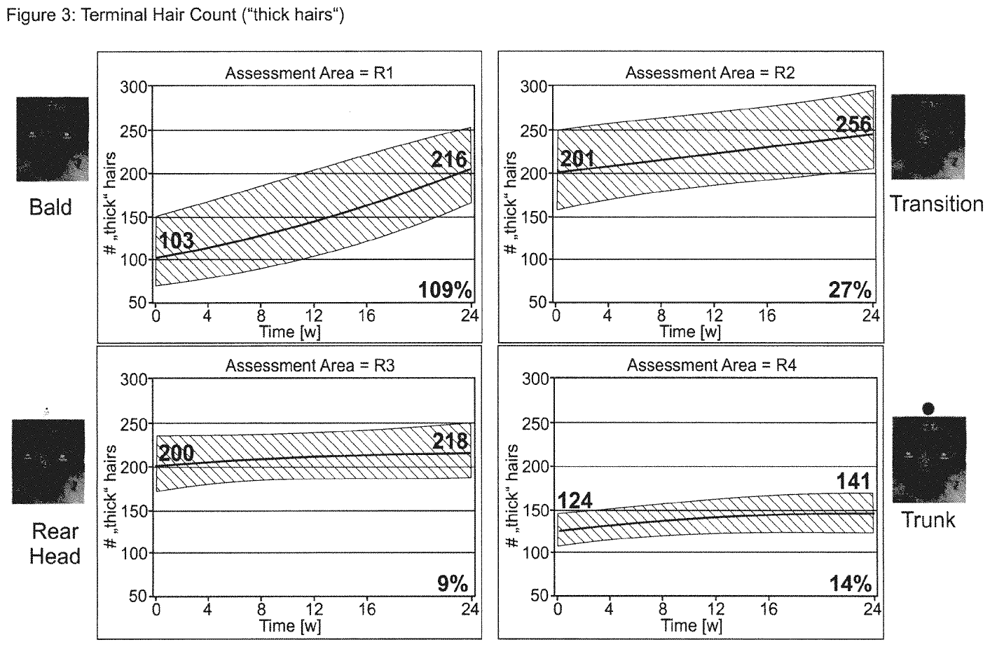

FIG. 3 represents an analysis of terminal hair counts in the “bald” (A), “transition” (B), “rear head” (C) and “trunk” (D) areas over the treatment period of 24 weeks. Such areas were predefined at baseline and marked by 2 tattoos which occurred in the upper left and lower right corner of the trichoscan image and allowed to monitor the very same hair follicles over the entire study duration. A drastic increase of the terminal hair count in bald areas (109% increases) was demonstrated as well as an increase in transition areas (27%). Expectedly, minor changes occurred in the rear head and trunk areas where few vellus hairs but many terminal hairs were present already before treatment start.

1. A method for treatment of male and female pattern hair loss, comprising administering to a patient in need thereof a pharmaceutically effective amount of a stable antibody-containing liquid or lyophilized formulation, wherein the formulation comprises:
20-120 mg/mL antibody, wherein the antibody is the PRLR antibody mat3;
10-50 mM arginine HCl;
5-30 mM histidine;
1-10 mM methionine;
50-150 ppm non-ionic surfactant; and
34-292 mM sugar.

2. The method according to claim 1, wherein the non-ionic surfactant is polysorbate and the sugar is sucrose.

3. The method according to claim 1, wherein the formulation comprises:
30 mM L-arginine HCl;
1.8 mM L-histidine;
8.2 mM L-histidine HCl;
5 mM L-methionine;
80 ppm polysorbate 80; and
234 mM sucrose.

4. The method according to claims claim 1, wherein the antibody concentration is 60 mg/mL.

5. The method according to any proceeding claims claim 1, wherein the PRLR antibody mat 3 comprises an antigen binding domain, wherein said antigen binding domain comprises a heavy chain variable region and a light chain binding variable region, wherein
a. the amino acid sequences of the HCDR1, HCDR2, and HCDR3 of said heavy chain variable region are selected from the group consisting of the amino acid sequence of SEQ ID NO: 3, 4, and 5 ; and
b. the amino acid sequences of the LCDR1, LCDR2, and LCDR3 of said light chain variable region are selected from the group consisting SEQ ID NO: 6, 7, and 8.

6. The method according to claim 1, wherein the PRLR antibody mat 3 comprises an antigen binding domain, wherein said antigen binding domain comprises a heavy chain variable region and a light chain binding variable region, wherein the amino acid sequence of the LCDR1 of said light chain variable region is SEQ ID NO: 9.

7. The formulation method according to any proceeding claims claim 1, wherein the PRLR antibody comprises a heavy chain variable region and a light chain binding variable region, wherein
a. the amino acid sequence of said heavy chain variable region is to SEQ ID NO: 1, and
b. the amino acid sequence of said light chain variable region is to SEQ ID NO: 2.

8. The method according to claim 4, wherein the formulation has having a pH value in the range from 5.0 to 6.5.

9. The method according to claim 5, wherein the formulation is stable for at least 6 months.

10. A method for treatment of prolactin₌related forms of hair loss caused by prolactinoma, comprising administering to a patient in need thereof a pharmaceutically effective amount of a stable antibody-containing liquid or lyophilized formulation, wherein the formulation comprises:
20-120 mg/mL antibody, wherein the antibody is the PRLR antibody mat3;
10 -50 mM arginine HCl;
5-30 mM histidine;
1-10 mM methionine;
50-150 ppm non-ionic surfactant; and
34-292 mM sugar.

11. A method for treatment of prolactin-related forms of hair loss caused by neuroleptics, comprising administering to a patient in need thereof a pharmaceutically effective amount of a stable antibody-containing liquid or lyophilized formulation, wherein the formulation comprises:
20-120 mg/mL antibody, wherein the antibody is the PRLR antibody mat3;
10 -50 mM arginine HC1;
5- 30 mM histidine;
110 mM methionine;
50-150 ppm non-ionic surfactant; and
34-292 mM sugar.

12. A method for treatment of postpartal hair loss, comprising administering to a patient in need thereof a pharmaceutically effective amount of a stable antibody-containing liquid or lyophilized formulation, wherein the formulation comprises:
20-120 mg/mL antibody, wherein the antibody is the PRLR antibody mat3;
10-50 mM arginine HCl;
5-30 mM histidine;
1-10 mM methionine;
50-150 ppm non-ionic surfactant; and
34-292 mM sugar.
